(19) United States
(12) Patent Application Publication
(10) Pub. No.: US 2022/0251052 A1
(43) Pub. Date: Aug. 11, 2022

(54) IMPROVED PROCESS FOR THE PREPARATION OF 2-(3,5-DICHLOROPHENYL)-1,3-BENZOXAZOLE-6-CARBOXYLIC ACID OR ITS PHARMACEUTICALLY ACCEPTABLE SALTS AND POLYMORPHS THEREOF

(71) Applicant: MSN LABORATORIES PRIVATE LIMITED, R&D CENTER, Sangareddy (Dist), Hyderabad, Telangana (IN)

(72) Inventors: Thirumalai Rajan SRINIVASAN, HYDERABAD, TELANGANA (IN); Eswaraiah SAJJA, HYDERABAD, TELANGANA (IN); Venkat Reddy GHOJALA, HYDERABAD, TELANGANA (IN); Rajeshwar Reddy SAGYAM, HYDERABAD, TELANGANA (IN); Markandeya BEKKAM, HYDERABAD, TELANGANA (IN)

(73) Assignee: MSN LABORATORIES PRIVATE LIMITED, R&D CENTER, Sangareddy (Dist), Hyderabad, Telangana (IN)

(21) Appl. No.: 17/624,591

(22) PCT Filed: Jul. 4, 2020
(86) PCT No.: PCT/IN2020/050584

§ 371 Date: Jan. 4, 2022

(30) Foreign Application Priority Data
Jul. 4, 2019 (IN) 201941026908
Feb. 10, 2020 (IN) 202041005729

(51) Int. Cl. C07D 263/57; C07C 213/08

(52) U.S. Cl. CPC C07D 263/57; C07B 2200/13; C07C 213/08

(57) ABSTRACT

The present invention relates to process for the preparation of 2-(3,5-dichlorophenyl)-1,3- benzoxazole-6-carboxylic acid of formula (I), its pharmaceutically acceptable salts and polymorphs thereof which are used in the treatment of cardiomyopathy of wild type or hereditary transthyretin-mediated amyloidosis.

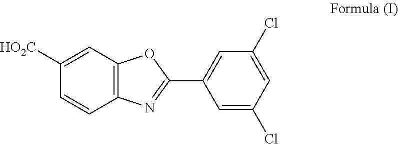 [SMILES: O=C(O)c1ccc2nc(-c3cc(Cl)cc(Cl)c3)oc2c1]

### Related applications

This patent application claims the benefit of priority of Indian patent application bearing number IN 201941026908 filed on 4 Jul., 2019 which is incorporated herein by reference in its entirety.

### Field of the invention

The present invention provides novel polymorphs of 2-(3,5-dichlorophenyl)-1,3- benzoxazole-6-carboxylic acid of formula (I) and process for preparation thereof,

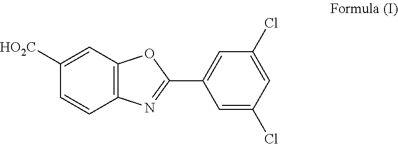 [SMILES: O=C(O)c1ccc2nc(-c3cc(Cl)cc(Cl)c3)oc2c1]

The present invention also provides an improved process for the preparation of formula (I) or its pharmaceutically acceptable salts thereof.

The present invention further provides amorphous premix of 2-(3,5-dichlorophenyl)- 1,3-benzoxazole-6-carboxylic acid (1-deoxy- 1 -methylamino-D-glucitol also called as “N- methyl-D-glucamine”) of formula (Ia) with one or more pharmaceutically acceptable excipients. The chemical structure of formula (Ia) is shown below:

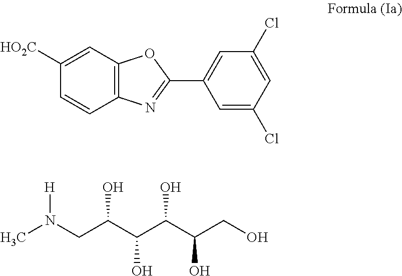 [SMILES: O=C(O)c1ccc2nc(-c3cc(Cl)cc(Cl)c3)oc2c1 ; CNC[C@H](O)[C@@H](O)[C@H](O)[C@H](O)CO]

### BACKGROUND OF THE INVENTION

2-(3,5-Dichlorophenyl)-1,3-benzoxazole-6-carboxylic acid commonly known as “Tafamidis” and its meglumine salts are both approved under the brand names Vyndamax® and Vyndaqel® respectively by USFDA on 3 May, 2019 to Foldrx Pharmaceuticals Inc. Vyndaqel® and Vyndamax® both indicated for the treatment of the cardiomyopathy of wild type or hereditary transthyretin-mediated amyloidosis in adults to reduce cardiovascular mortality and cardiovascular-related hospitalization. Vyndaqel® available in 20 mg soft gelatin capsules, each soft capsule contains 20 mg of micronized Tafamidis meglumine equivalent to 12.2 mg Tafamidis. Vyndamax® available in 61 mg of Tafamidis soft gelatin capsule orally once daily.

US 7214695 B2 discloses Tafamidis and its pharmaceutically acceptable salts and also process for the preparation comprising reaction of 4-amino-3-hydroxybenzoic acid with 3,5- dichlorobenzoyl chloride in presence of tetrahydrofuran and pyridine to provide 4-(3,5- dichlorobenzamido)-3-hydroxybenzoic acid which is reacted with p-toluenesulfonic acid mono hydride in presence of xylene and reflux to provide crude Tafamidis. The obtained compound was dissolved in methanol-benzene and reacted with trimethylsilyldiazomethane to provide methyl ester which is further hydrolyzed to provide Tafamidis.

US 9249112 B2 discloses crystalline form, liquid crystal form and amorphous form of Tafamidis meglumine. This patent also discloses a process for the preparation of amorphous form of Tafamidis meglumine by melting.

US 9770441 B1 discloses crystalline Form 1, Form 2, Form 4, Form 6 and amorphous of Tafamidis of formula (1) and its preparation comprising reaction of 4-amino-3- hydroxybenzoic acid with 3,5-dichlorobenzoylchloride to provide crude 4-[(3,5- dichlorobenzoyl)amino]-3-hydroxybenzoic acid which is further isolated after work up using triethylamine. The obtained compound was further cyclized to provide Tafamidis.

The main drawback of the above prior art processes is the use of bases like triethylamine and pyridine which causes generation of tetrachloroamide impurity in crude 4- [(3,5-dichlorobenzoyl)amino]-3-hydroxybenzoic acid.

The chemical structure of tetrachloroamide impurity is as follows:

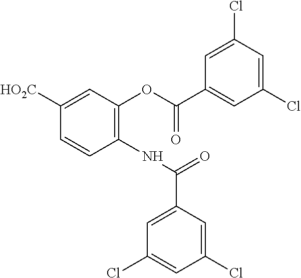 [SMILES: O=C(O)c1ccc(NC(=O)c2cc(Cl)cc(Cl)c2)c(OC(=O)c2cc(Cl)cc(Cl)c2)c1]

This impurity remained in 4-[(3,5-dichlorobenzoyl)amino]-3-hydroxybenzoic acid intermediate compound up to about 2-3%. Hence, there is required different purification methods to eliminate the said impurity which is tedious, solvent consuming and time consuming process which increases the cost of the production.

US 20190119226 Al discloses crystalline Form-E of Tafamidis meglumine and process for its preparation thereof.

Drug substances with different polymorphic forms having desirable pharmacokinetic properties often, solubility, dissolution, flow rate and bioavailability. Hence, there is need in developing strategies for improving the pharmaceutical properties of drug substances with different polymorphic forms. Further there is a significant need in the art to develop a stable form of Tafamidis or its meglumine salt which is simple, cost effective and liable for industrial scale preparation methods and also suitable for pharmaceutical composition.

Inventors of the present invention developed novel crystalline forms of Tafamidis of formula (I).

Inventors of the present invention also developed an amorphous premix of Tafamidis meglumine salt of formula (Ia) with one or more pharmaceutically acceptable excipients and also process for its preparation thereof.

Inventors of the present invention developed a novel process for the preparation of Tafamidis of formula (I).

Inventors of the present invention arrest the tetrachloroamide impurity by conducting the reaction of 4-amino-3-hydroxybenzoic acid with 3,5-dichlorobenzoyl chloride in the absence of a base.

Advantages of the present invention:

Absence of a base during the reaction or later work-up of obtained intermediate compound 4-[(3,5-dichlorobenzoyl)amino]-3-hydroxybenzoic acid provides substantially free from tetrachloroamide impurity.

Novel crystalline forms of Tafamidis

Brief description of the invention

In first embodiment, the present invention provides a novel crystalline form of Tafamidis of formula (I) (hereinafter designated as “Form-S”) and also its process for preparation thereof.

In second embodiment, the present invention provides a novel crystalline form of Tafamidis of formula (I) (hereinafter designated as “Form-N”) and also its process for preparation thereof.

In third embodiment, present invention provides a novel crystalline form of Tafamidis of formula (I) (hereinafter designated as “Form-R”) and also its process for preparation thereof.

In fourth embodiment, the present invention provides amorphous premix of Tafamidis meglumine of formula (Ia) with one or more pharmaceutically acceptable excipients and process for its preparation thereof.

In fifth embodiment, the present invention provides a process for the preparation of amorphous form of Tafamidis meglumine of formula (Ia).

In sixth embodiment, the present invention provides an improved process for the preparation of Tafamidis of formula (I) or its pharmaceutically acceptable salts.

In seventh embodiment, the present invention provides a novel process for the preparation of Tafamidis of formula (I) or its pharmaceutically acceptable salts.

In eighth embodiment, present invention provides pharmaceutical composition com- prising crystalline forms selected from Form-S, Form-N and Form-R of Tafamidis of formula (I) and one or more pharmaceutically acceptable excipients.

### BRIEF DESCRIPTION OF THE FIGURES

FIG. 1: Illustrates Powder X-Ray Diffraction pattern of crystalline Form-S of Tafamidis obtained according to Example 3.

FIG. 2: Illustrates Powder X-Ray Diffraction pattern of crystalline Form-N of Tafamidis obtained according to Example 4.

FIG. 3: Illustrates Powder X-Ray Diffraction pattern of crystalline Form-R of Tafamidis obtained according to Example 5.

FIG. 4: Illustrates Powder X-Ray Diffraction pattern of amorphous premix of Tafamidis meglumine with PVP obtained according to Example 7.

FIG. 5: Illustrates Powder X-Ray Diffraction pattern of amorphous premix of Tafamidis meglumine with HPMC obtained according to Example 8.

FIG. 6: Illustrates Powder X-Ray Diffraction pattern of amorphous premix of Tafamidis meglumine with HPC obtained according to Example 9.

Fig.7: Illustrates Powder X-Ray Diffraction pattern of amorphous premix of Tafamidis meglumine with MCC obtained according to Example 10.

FIG. 8: Illustrates Powder X-Ray Diffraction pattern of amorphous premix of Tafamidis meglumine with HPMC-AS obtained according to Example 11.

FIG. 9: Illustrates Powder X-Ray Diffraction pattern of amorphous premix of Tafamidis meglumine with Syloid obtained according to Example 12.

FIG. 10: Illustrates Powder X-Ray Diffraction pattern of amorphous premix of Tafamidis meglumine with co-povidone obtained according to Example 13.

FIG. 11: Illustrates Powder X-Ray Diffraction pattern of amorphous form of Tafamidis meglumine obtained according to Example 14.

### DETAILED DESCRIPTION OF THE INVENTION

The term “solvent” used in the present invention refers to “hydrocarbon solvents” selected from n-hexane, n-heptane, cyclohexane, petroleum ether, benzene, toluene, pentane, cycloheptane, methyl cyclohexane, ethylbenzene, m-, o-, or p-xylene, or naphthalene and mixture thereof; “ether solvents” selected from dimethoxymethane, tetrahydrofuran, 1,3- dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, 1,2-dimethoxy ethane and mixture thereof; “ester solvents” selected form methyl acetate, ethyl acetate, isopropyl acetate, n-butyl acetate, isobutyl acetate and mixture thereof; “polar-aprotic solvents selected form dimethylacetamide (DMA), dimethylformamide (DMF), dimethylsulfoxide (DMSO), N- methylpyrrolidone (NMP) and mixture thereof; “chloro solvents” selected form dichloromethane, dichloroethane, chloroform, carbon tetrachloride and mixture thereof; “ketone solvents” selected form acetone, methyl ethyl ketone, methyl isobutylketone and mixture thereof; “nitrile solvents” selected form acetonitrile, propionitrile, isobutyronitrile and mixture thereof; “alcohol solvents” selected form methanol, ethanol, n-propanol, isopropanol, n-butanol, isobutanol, t-butanol, 2-nitroethanol, 2-fluoroethanol, 2,2,2-trifluoroethanol, ethylene glycol, 1,2-propanediol (propylene glycol), 2-methoxyethanol, 1, 2-ethoxyethanol, diethylene glycol, 1,2, or 3-pentanol, neo-pentyl alcohol, t-pentyl alcohol, diethylene glycol monoethyl ether, cyclohexanol, benzyl alcohol, phenol, or glycerol and mixture thereof; “polar solvents” selected from water or mixtures thereof.

The term “base” used in the present invention refers to inorganic bases selected form “alkali metal carbonates” selected form sodium carbonate, potassium carbonate, lithium carbonate and mixture thereof; “alkali metal bicarbonates” selected from sodium bicarbonate, potassium bicarbonate mixture thereof; “alkali metal hydroxides” selected from sodium hydroxide, potassium hydroxide, lithium hydroxide and mixture thereof; alkali metal hydrides selected form sodium hydride, potassium hydride, lithium hydride and mixture thereof; alkali metal amides selected form sodium amide, potassium amide, lithium amide; organic bases selected form triethylamine, isopropyl ethylamine, diisopropyl amine, diisopropylethylamine, piperidine, pyridine, tributyl amine, 4-dimethylaminopyridine, N-methyl morpholine and/or mixtures thereof.

The term “acid” used in the present invention refers to inorganic acids selected form hydrochloric acid (HC1), hydrobromic acid (HBr), hydroiodic acid (HI), sulfuric acid (H2SO4); organic acids selected form acetic acid, methanesulfonic acid, p-toluenesulfonic acid, trifluoroacetic acid.

The term “coupling agent” used in the present invention selected from but not limited to 1,1′-carbonyl diimidazole (CDI), N,N′-dicyclohexylcarbodiimide (DCC), N,N′- diisopropyl carbodiimide (DIC), 1-ethyl-3-(3-dimethylaminopropyl)carbodiimide HC1 (EDC .HC 1), 1-[bis(dimethylamino)methylene]-1H-1,2,3 -triazolo[4, 5 -b]pyridinium3 -oxid hexafluoro phosphate (HATU), 2-(1H-benzotriazol-1-yl)-1,1,3,3-tetramethyluronium hexafluoro phosphate (HBTU), alkyl/aryl haloformates selected from but not limited to ethyl chloroformate, benzylchloroformate; diphenylphosphoroazidate (DPPA), thionyl chloride, oxalyl chloride, phosphorous oxychloride, phosphorous pentachloride, 4- methyl-2-oxopentanoyl chloride (iBuCOCOC1), (benzotriazol-l-yloxy) tris(dimethylamino) phosphoniumhexafluorophosphate(BOP), benzotriazol-l-yl-oxy tripyrrolidinophosphonium hexafluorophosphate (PyBOP), methane sulfonyl chloride, p-toluenesulfonyl chloride and mixture thereof.

The term “protecting group” is selected from but not limited to a group of reagents independently selected such that they are capable of protecting the hydroxy or amine groups of various compounds of the present invention selected from benzyloxycarbonyl (Cbz), 2,2,2-trichloroethoxy carbonyl(Troc), 2- (trimethyl silyl)ethoxycarbonyl (Teoc), 2-(4-trifluoromethylphenyl sulfonyl) ethoxycarbonyl (Tsc), 1 -adamantyloxycarbonyl (Adoc), 2-adamantylcarbonyl(2-Adoc), 2,4-dimethylpent-3 - yloxycarbonyl (Doc), cyclohexyloxy carbonyl(Hoc), 1, 1-dimethyl-2,2,2- trichloroethoxycarbonyl (TcBOC), vinyl, 2-chloroethyl, 2-phenylsulfonylethyl, allyl, benzyl, 2-nitrobenzyl, 4-nitrobenzyl, diphenyl-4-pyridylmethyl, N′,N′-dim ethylhydrazinyl, methoxymethyl, t-butoxymethyl (Bum), benzyloxymethyl (BOM), 3,4-dihydro-2H- pyranyl(DHP), tetrahydropyranyl (THP), tetrahydrofuranyl (THF), methyl, ethyl, acetyl, benzyl, benzoyl, benzyloxycarbonyl (Cbz), trifluoroacetyl, pivaloyl, allyl, methoxymethyl (MOM), ethoxyethy(EE), methoxyethoxymethyl (MEM), p-methoxybenzyl (PMB), methylthiomethyl (MTM), triphenylmethyl (trityl), methoxytrityl (MMT), dimethoxytrityl (DMT), benzyloxymethyl (BOM), tert.butoxy carbonyl (Boc) and mixture thereof; the preferable silyl protecting groups can be selected from but not limited to trialkylsilyl, triarylsilyl, alkyl/aryl silyl selected from trimethylsilyl (TMS), triethylsilyl (TES), triisopropylsilyl (TIPS), tert.butyldimethylsilyl (TB S or TBDMS), tri-iso- propylsilyloxymethyl (TOM), tert-butyldiphenylsilyl (TBDPS) and thereof,

The term “protecting agent” is selected from but not limited to trialkyl silyl halides selected form trimethylsilyl chloride (TMSC1), triethylsilyl chloride (TESC1), triisopropylsilyl chloride (TIPSC1), tert-butyldimethylsilyl chloride (TBDMSC1), tert- butyldiphenylsilyl chloride (TBDPSC1) and thereof; trialkyl silyl triflates selected form trimethylsilyl triflate(TMSOTf), triethylsilyl triflate (TESOTf), triisopropylsilyl triflate (TIPSOTf), tert-butyldimethyl silyl triflate (TBDMSOTf or TBSOTf), tert-butyldiphenylsilyl triflate(TBDPSC1) thereof; N,0-bi s(trimethylsilyl)acetamide (BSA), hexemethyldisilazane (HMDS), fluorenylmethyloxy carbonyl chloride (FMOC chloride), dihydropyran, 2-chloro tetrahydrofuran, diazomethane, methyl halides, acetyl chloride, acetic anhydride, benzyl halides, benzoyl chloride, benzoic anhydride, benzyloxycarbonyl chloride, trifluoroacetyl chloride, trifluoroacetic anhydride, trifluoroacetic acid, alkyl trifluoroacetates selected from methyl trifluoroacetate, ethyl trifluoroacetate, isopropyl trifluoroacetate, vinyl trifluoroacetate; and thereof; tert-butyl acetyl chloride, tert-butyl acetic anhydride, allyl halides, methoxymethyl halides, ethoxyethyl halides, methoxyethoxymethyl halides, p- methoxybenzyl halides, methylthiomethyl halides, trityl halides, benzyloxymethyl halides, di- tert.butyl dicarbonate (DIBOC), alkyl/aryl sulfonic acids/acid halides/anhydrides selected form methanesulfonyl chloride, ethanesulfonyl chloride, benzenesulfonyl chloride, toluenesulfonyl chloride, methanesulfonic acid, ethanesulfonic acid, p-toluenesulfonic acid, p-toluenesulfonic anhydride and mixture thereof.

The term “deprotecting agent” can be selected based on the protecting group employed. The deprotecting agent can be selected from but not limited to acids selected form hydrochloric acid, hydrobromic acid, phosphoric acid, acetic acid, formic acid, trifluoroacetic acid, methane sulfonic acid, p-toluene sulfonic acid, camphor sulfonic acid and thereof, bases selected form alkali metal hydroxides, alkali metal carbonates, cesium carbonate/imidazole, alkali metal bicarbonates, ammonia, cerium ammonium nitrate (CAN), 1,8-diazabicyclo[5.4.0]undec-7-ene (DBU), hydrogenating agents selected from Pd, Pd/C, Pd(OH)2/C (Pearlman's catalyst), palladium acetate, platinum oxide (PtO2), platinum black, sodium borohydride, BF3-etherate, Raney-Ni, tri ethyl silane, trimethyl silyl halides, copper(II) chloride dihydrate and thereof.

The term “chlorinating agent” is selected from the group consisting of but is not limited to sodium hypochlorite (NaOC1), thionyl chloride (SOC12), phosphorus trichloride (PC13), phosphorus pentachloride (PC15), phosphorus oxychloride (POC13), oxalyl chloride, CC14, N-chlorosuccinimide (NCS), C12 gas, sulfuryl chloride, or combination thereof

The term “premix” refers to mixture of Tafamidis meglumine salt and at least one pharmaceutically acceptable excipient.

The term “pharmaceutically acceptable salts” described hereinbefore are obtained by reacting Tafamidis with acids selected from hydrochloric acid, hydrobromic acid, sulfuric ac- id, nitric acid, phosphoric acid, methanesulfonic acid, ethanesulfonic acid, p-toluenesulfonic acid, salicylic acid and thereof. In some instances, pharmaceutically acceptable salts are ob- tained by reacting Tafamidis described herein with a base to form a salt selected from an am- monium salt, an alkali metal salt, inorganic salts selected from a sodium or a potassium salt, an alkaline earth metal salt, selected from a calcium or a magnesium salt, a salt of organic b ses selected from dicyclohexylamine, N-methyl-D-glucamine, tris(hydroxymethyl) methyla- mine, and salts with amino acids selected from arginine, lysine, and thereof.

As used herein, the term “substantially free” refers to a compound of the present invention having one or more impurities less than about 0.5% or less than about 0.4% or less than about 0.3% or less than about 0.2% or less than about 0.1% or less than about 0.05% or below detection level.

As used herein, the term “pure” refers to Tafamidis has a purity greater than about 99.50% by or greater than about 99.70% or greater than about 99.80% by HPLC.

As used herein, the term “stable” refers to Tafamidis crystalline Form-R that substan- tially does not convert to any other polymorphic form.

As used herein, the term “about” refers to 0.0001-5% deviation from the stated num- ber or range of numbers described in the specification.

In first embodiment, present invention provides crystalline Form-S of Tafamidis of formula (I).

In first aspect of first embodiment, crystalline Form-S of Tafamidis of formula (I) is characterized by its Powder X-Ray Diffraction (PXRD) pattern having peaks at about 10.7 and 14.5 ±0.2 degrees two-theta. Crystalline Form-S of the present invention is further char- acterized by its X-Ray powder diffraction pattern having additional peaks at about 10.1 and 16.8 ±0.2 degrees two-theta. The crystalline Form-S of Tafamidis of formula (I) is further characterized by the PXRD pattern as illustrated in figure-1.

In second aspect of first embodiment, crystalline Form-S of Tafamidis of formula (I) of the present invention having the acetic acid content in range of about 5% to about 25%, or about 10% to about 20% or about 14% to about 18%.

In third aspect of first embodiment, the present invention provides a process for the preparation of crystalline Form-S of Tafamidis of formula (I), comprising:
a) providing a mixture of Tafamidis in acetic acid,
b) isolating crystalline Form-S of Tafamidis
   wherein the mixture in step-a) can be heterogeneous or homogeneous in acetic acid and stirred for at least about from 30 min to about 15 hrs; prefarebly about 1 to about 5 hours; the term “isolating” in step-b) refers to filtration or distillation or decantation from the mixture.

In second embodiment, the present invention provides crystalline Form-N of Tafamid- is of formula (I).

In first aspect of second embodiment, crystalline Form-N of formula (I) is character- ized by Powder X-Ray Diffraction (PXRD) pattern having peaks at about 10.1 and 22.9 ±0.2 degrees two-theta. The crystalline Form-N of Tafamidis of formula (I) is further characterized by the PXRD pattern as illustrated in FIG. 2.

In second aspect of second embodiment, crystalline Form-N of Tafamidis of formula (I) of the present invention having the water content in range about 2% to 14%, or about 4% to about 12% or about 6% to about 10%.

In third aspect of second embodiment, present invention provides a process for the preparation of crystalline Form-N of Tafamidis of formula (I), comprising:
a) providing a mixture of crystalline Form-S of Tafamidis of formula (I) in water,
b) isolating crystalline Form-N of Tafamidis of formula (I)
   wherein the mixture in step-a) can be a heterogeneous or homogeneous in water and stirred for at least about from 30 min to about 10 hrs; prefarebly about 1 to about 5 hours; the term “isolating” in step-b) refers to filtration or distillation or decantation from the mixture.

In third embodiment, the present invention provides crystalline Form-R of Tafamidis formula (I).

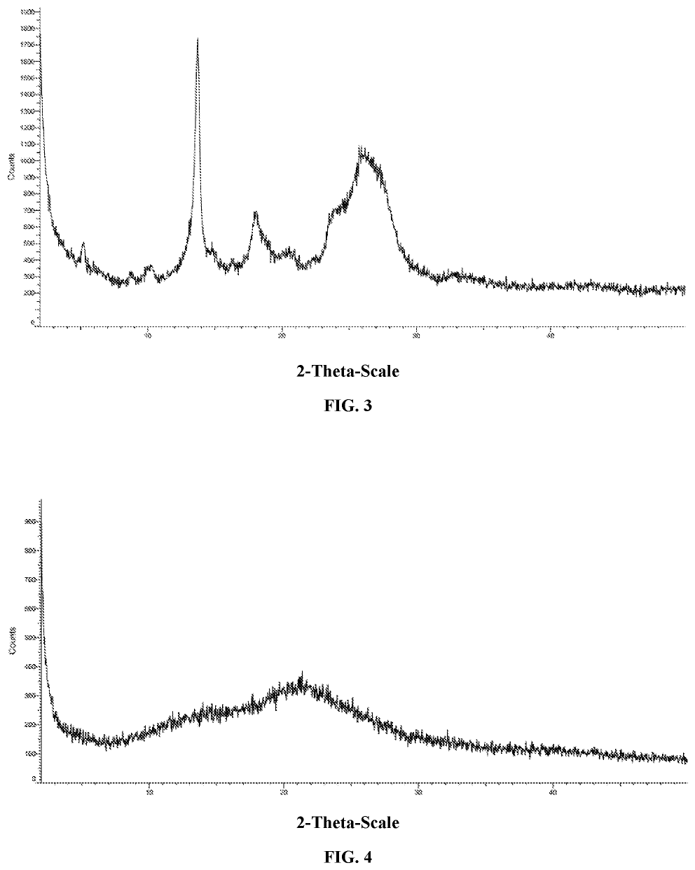

In first aspect of third embodiment, crystalline Form-R of Tafamidis of formula (I) characterized by Powder X-Ray Diffraction (PXRD) having peaks at about 5.2 and 13.7 ±0.2 degrees two-theta. Crystalline Form-R of the present invention is further characterized by its X-Ray powder diffraction pattern having additional peak at about 18.0 ±0.2 degrees two- theta. The crystalline Form-R of Tafamidis of formula (I) is further characterized by the PXRD pattern as illustrated in FIG. 3.

In second aspect of third embodiment, crystalline Form-R of Tafamidis of formula (I) of the present invention is anhydrous or contains less than about 2% or less than about 1.5% or less than about 1% or less than about 0.5% of water content.

In third aspect of third embodiment, crystalline Form-R of Tafamidis of formula (I) is stable under stress conditions at 60±5° C. for 24 hours, 10 tons of pressure, under UV light at 254nm for 24 hours and higher humidity conditions of 75±5% relative humidity for 24 hours.

In third aspect of third embodiment, the present invention provides a process for the preparation of crystalline Form-R of Tafamidis of formula (I), comprising:
a) providing a mixture of Tafamidis of formula (I) in a first solvent.
b) combining the mixture obtained in step-a) with second solvent,
c) isolating crystalline Form-R of Tafamidis of formula (I)
   wherein the first solvent used in step-a) is polar aprotic solvent selected from dimethylsulfox- ide, dimethyl formamide, dimethylacetamide thereof; second solvent in step-b) is water; the term “combining” refers to adding the mixture obtained in step-a) to anti-solvent or vice- versa; “isolating” in step-c) refers to slurrying/stirring the obtained compound with acetic acid and water followed by filtration and drying. Drying may be suitably carried out using equip- ment such as a vaccum tray dryer, atmospheric tray dryer, rotary cone vacuum dryer, vacuum oven, air oven, fluidized bed dryer, spin flash dryer, flash dryer, and thereof, at atmospheric pressure or under reduced pressure. Drying may be carried out at temperatures less than about 100° C., less than about 75° C., less than about 50° C., less than about 25° C., or any other suitable temperatures optionally in the presence or absence of air or an inert atmosphere such as nitro- gen, argon, neon, or helium or optionally under vaccum. The drying may be carried out for any desired time periods to achieve a desired purity of the product, such as, for example, from about 1 hour to about 15 hours, or longer or shorter.

In fourth aspect of third embodiment, the present invention provides a process for the preparation of crystalline Form-R of Tafamidis of formula (I), comprising drying of crystal- line Form-N of Tafamidis of formula (I).

wherein the drying conditions are same as defined hereinbefore.

In fourth embodiment, the present invention provides amorphous premix of Tafamidis meglumine of formula (Ia) with one or more pharmaceutically acceptable excipients

wherein the pharmaceutically acceptable excipient can be one or more selected from pov- idone, co-povidone, ethyl cellulose, hydroxypropylcellulose, hydroxypropyl methylcellulose, polyethylene glycol, soluplus, starch, microcrystalline cellulose, cross povidone, methylcellu- lose, cellulose ethers, sodium carboxymethylcellulose, dextrose, lactose, lactose monohydrate, sucrose, sorbitol, mannitol, polyvinylpyrrolidone, polyacrylamides, polyvinyloxoazolidone, polyvinylalcohols, carmellose, carmellose sodium, glycerol monosterate, magnesium alumi- nometa silicate or starch and syloid.

In first aspect of fourth embodiment, the present invention provides a process for the preparation of the amorphous premix of Tafamidis meglumine of formula (Ia), comprising:
a) providing a solution comprising Tafamidis meglumine of formula (Ia) and one or more pharmaceutically acceptable excipients in a solvent,
b) removing the solvent from the mixture obtained in step-a) to get amorphous premix of Tafamidis meglumine of formula (Ia)
   wherein the solvent and pharmaceutically acceptable excipient used in step-a) is same as de- fined hereinbefore; the term “removing the solvent” in step-b) refers to spray drying, distilla- tion under vacuum, evaporation, lyophilization, decantation and centrifugation technics and like.

In fifth embodiment, the present invention provides a process for the preparation of amorphous form of Tafamidis meglumine of formula (Ia), comprising:
a) providing a solution of Tafamidis meglumine in a solvent,
b) removing the solvent from the mixture to get amorphous premix of Tafamidis meglu- mine of formula (Ia)
   wherein the solvent in step-a) is water; wherein removing the solvent using one or more of spray drying, lyophilization, distillation, distillation under vacuum, evaporation, decantation, and centrifugation technics.

In sixth embodiment, the present invention provides an improved process for the prep- aration of Tafamidis of formula (I) or its pharmaceutically acceptable salts, comprising:
a) reacting 3,5-dichlorobenzoyl chloride with 4-amino-3-hydroxybenzoic acid to provide 4-(3,5-dichlorobenzamido)-3-hydroxybenzoic acid in the absence of a base optionally in a solvent,
b) converting 4-(3 , 5 -dichlorobenzamido)-3 -hydroxybenzoic acid to Tafamidis of formula

(I) or its pharmaceutically acceptable salts.

wherein the solvent used in step-a) is selected form tetrahydrofuran, toluene & water or mix- tures thereof.

In seventh embodiment, the present invention provides a novel process for the prepa- ration of Tafamidis of formula (I) or its pharmaceutically acceptable salts, comprising:
a) chlorinating benzonitrile of formula (VI) to provide 3,5-dichcloro benzonitrile of for- mula (V),

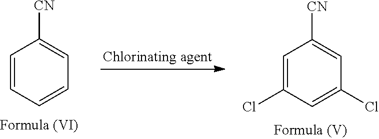 [SMILES: N#Cc1ccccc1 ; N#Cc1cc(Cl)cc(Cl)c1]

b) converting the compound of formula (V) to methyl 3,5-dichlorobenzimidate of formula (IV) or its salt,

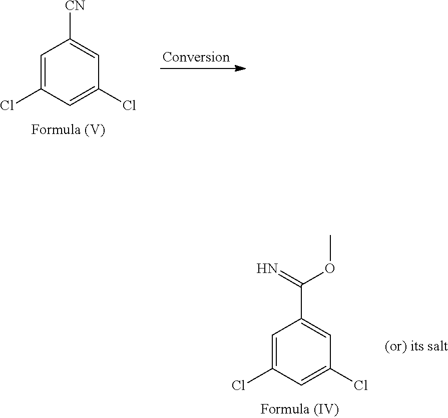

c) reacting the compound of formula (IV) or its salt with 4-amino-3-hydroxybenzoic acid or ester of formula (III) to provide compound of formula (II),

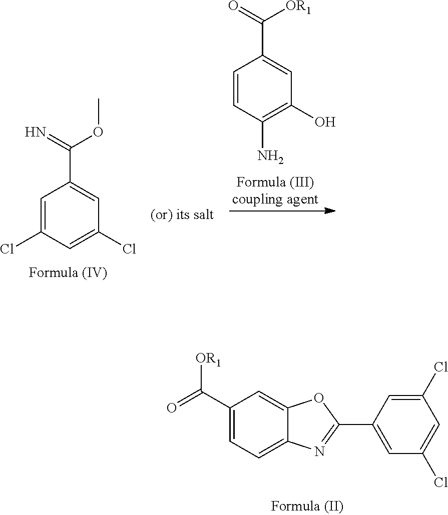

wherein “Ri” refers to hydrogen or Ci-4 alkyl group
d) converting the compound of formula (II) (wherein, Ri refers to Ci-4 alkyl group) to Tafamidis of formula (I) or its pharmaceutically acceptable salts thereof.

In eighth embodiment, present invention provides a pharmaceutical composition com- prising one or more crystalline forms selected from Form-S, Form-N and Form-R of Tafamid- is of formula (I) and one or more pharmaceutically acceptable excipients.

In one aspect of ninth embodiment, present invention preferably provides a pharma- ceutical composition comprising crystalline Form-R of Tafamidis of formula (I) and one or more pharmaceutically acceptable excipients.

Pharmaceutical compositions containing Tafamidis of formula (I) or its pharmaceuti- cally acceptable salts of the present invention may be prepared by using excipients selected from fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection prep- arations.

The oral pharmaceutical composition may contain one or more additional excipients selected from diluents, binders, disintegrants and lubricants. Exemplary diluents include lac- tose, sucrose, glucose, mannitol, sorbitol, calcium carbonate, microcrystalline cellulose, mag- nesium stearate and mixtures thereof. Exemplary binders are selected from L-hydroxy propyl cellulose, povidone, hydroxypropyl methyl cellulose, hydroxylethyl cellulose and pre- gelatinized starch.

Exemplary disintegrants are selected from croscarmellose sodium, cros-povidone, so- dium starch glycolate and low substituted hydroxylpropyl cellulose.

Exemplary lubricants are selected from sodium stearyl fumarate, magnesium stearate, zinc stearate, calcium stearate, stearic acid, talc, glyceryl behenate and colloidal silicon diox- ide. A specific lubricant is selected from magnesium stearate, zinc stearate, calcium stearate and colloidal silicon dioxide.

In yet another embodiment, crystalline Form-S, Form-N and Form-R of Tafamidis of formula (I) of the present invention can be micronized to achieve the desired better particle size distribution in order to make suitable formulation.

In yet another embodiment, crystalline Form-S, Form-N and Form-R of Tafamidis of formula (I) of the present invention are having particle size distribution D90 lessthan 200 ppm; preferably lessthan 100 ppm. More preferably lessthan 50 ppm or lessthan 20 ppm.

In yet another embodiment, crystalline Form-S, Form-N and Form-R of Tafamidis of formula (I) of the present invention can be further micronized or milled by 5 conventional techniques to get the desired particle size to achieve desired solubility profile based on different forms of pharmaceutical composition requirements. Techniques that may be used for particle size reduction include, but not limited to ball, roller and hammer mills, and jet mills. Milling or micronization may be performed before drying, or after the completion of drying of the product.

PXRD analysis of the crystalline forms, amorphous and amorphous premix of the present in- vention were carried out using BRUKER/AXS X-Ray diffractometer using Cu-Ka radiation of wavelength 1.5406 A° and at continuous scan speed of 0.03° /min.
High Performance Liquid Chromatography (HPLC) analysis of intermediate compound 4- (3,5-dichlorobenzamido)-3-hydroxybenzoic acid was carried out by following conditions: Apparatus: A liquid chromatograph is equipped with variable wavelength UV Detector; Col- umn: Inertsil ODS-2, 150x4.6 mm, 5μm; Flow rate: 1.0 mL/min; Wavelength: 230 nm; Col- umn temperature: 20° C.; Injection volume: 5μL; Elution: Gradient; Diluent: Acetonitrile and Water; Mobile phase-A: Accurately transfer 900 mL of Buffer and 100 mL of Acetonitrile into a 1000 mL; Mobile phase-B: Accurately transfer 900 mL of Acetonitrile and 100 mL of water into a 1000 mL.

The best mode of carrying out the present invention is illustrated by the below mentioned examples. These examples are for illustrative purposes only and in no way limit the scope of the present invention.

### EXAMPLES

Example-1: Preparation of 4-(3,5-dichlorobenzamido)-3-hydroxybenzoic acid

3,5-Dichlorobenzoic acid (149.67 gr) was added to dimethylformamide (5 ml) and tol- uene (300 ml) at 25-30° C. Thionyl chloride (200 ml) was added to the above mixture. Raised the temperature of the reaction mixture to 75-80° C. and stirred for 3 hrs. Distilled off the solvent from the reaction mixture under nitrogen atmosphere at 110-115° C. and co-distilled with toluene to get 3,5-dichlorobenzoyl chloride residue.

4-Amino-3-hydroxybenzoic acid (100 gr) was added to toluene (300 ml), tetrahydrofu- ran (200 ml) and water (200 ml) at 25-30° C. Cooled the mixture to 10-15° C. and stirred for 10-15 min. The above 3,5-dichlorobenzoyl chloride solution (dissolved in 100 ml of toluene) was added drop wise to the reaction mixture. Raised the reaction mixture temperature to 25- 30° and stirred for 2 hrs. Quenched the reaction mixture with methanol and distilled off the solvent under reduced pressure. Methanol (400 ml) and water (400 ml) were added to the ob- tained residue at 25-30° C. and stirred for 2 hrs at the same temperature. Filtered the precipitat- ed solid and washed with methanol. Methanol (800 ml) was added to the obtained wet materi- al and stirred for 2 hrs at 55-60° C. Cooled the temperature of the reaction mixture to 25-30° C. and stirred for 2 hrs. Filtered the precipitated solid, washed with methanol and then dried to provide title compound.

Yield: 179.0 gms, tetrachloroamide impurity: 0.01% by HPLC

Example-2: Preparation of 2-(3,5-dichlorophenyl)-1,3-benzoxazole-6-carboxylic acid (Tafamidis) of formula (I)

4-(3,5-Dichlorobenzamido)-3-hydroxybenzoic acid (250 gms) was added to toluene (2500 ml) at 25-30° C. and stirred for 15 min at same temperature. Basified the mixture with triethylamine (129.0 ml) at 25-30° C. and stirred for 60 min at same temperature. Methanesul- fonic acid was added to the reaction mixture at 25-30° C. and stirred for 14 hrs at 110-115° C. to provide the title compound

Yield: 167 gms

Example-3: Preparation of crystalline Form-S of Tafamidis of formula (I)

Tafamidis (10 gr) was added to acetic acid (200 ml) at 25-30° C. and stirred for 3 hrs. Filtered the solid and then dried to afford the title compound

Yield: 9.3 gr.

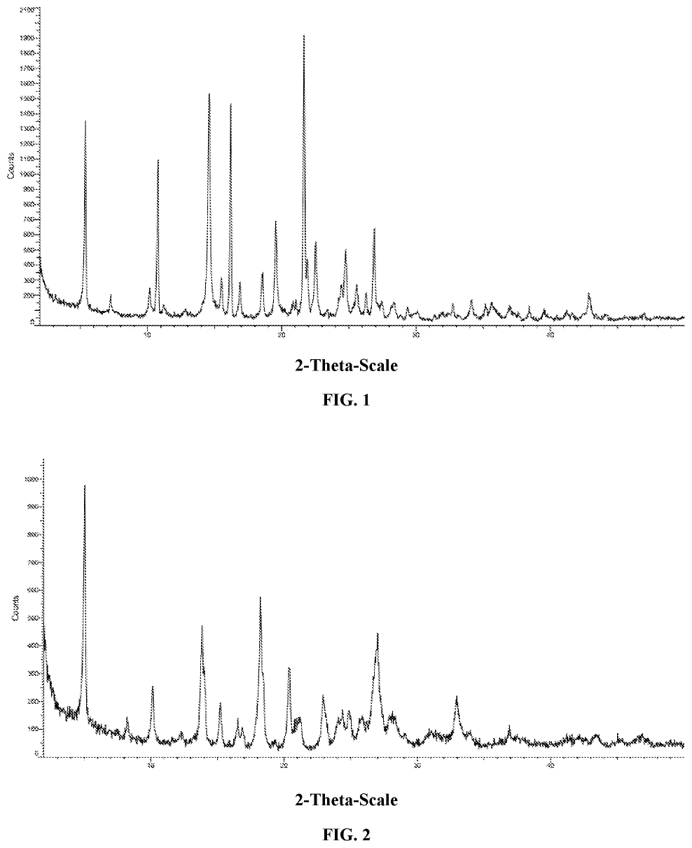

The PXRD pattern of the obtained crystalline Form-S of Tafamidis was illustrated in FIG.1.

Example-4: Preparation of crystalline Form-N of Tafamidis of formula (I)

Crystalline Form-S of Tafamidis (8 gr) was added to water (80 ml) at 25-30° C. and stirred for 2 hrs. Filtered the solid and then dried to get the title compound.

Yield: 8.1 gr.

The PXRD pattern of the obtained crystalline Form-N of Tafamidis was illustrated in FIG 2.

Example-5: Preparation of crystalline Form-R of Tafamidis of formula (I)

Crystalline Form-N of Tafamidis was dried at 80-85° C. for 2 hrs to afford the title compound

Yield: 6.2 gr.

The PXRD pattern of the obtained crystalline Form-R of Tafamidis was illustrated in FIG. 3.

Example-6: Preparation of crystalline Form-R of Tafamidis of formula (I)

Tafamidis (6 gr) was dissolved in dimethylsulfoxide (54 ml) at 70-75° C. Filtered the obtained solution. Water (120 ml) was added to the obtained filtrate at 50-55° C. and stirred. Cooled the reaction mixture to 25-30° C. and stirred. Filtered the compound and slurried in acetic acid (60 ml) followed by water (90 ml). Filtered the obtained compound and dried at about 50-55° C. for about 5 hrs to afford the title compound.

Yield: 4.9 gr.

The PXRD pattern of the obtained compound was similar to the PXRD of Form-R illustrated in FIG. 3.

Example-7: Preparation of amorphous premix of Tafamidis meglumine of formula (Ia) with polyvinyl pyrrolidine (PVP)

Tafamidis meglumine of formula (Ia) (0.5 gms) was dissolved in water (11 ml) at 55- 60° C. Aqueous polyvinyl pyrrolidine solution (0.5 gms of PVP dissolved in 3 ml of water) was added to the above obtained solution and distilled off the solvent under reduced pressure and then dried to get the title compound. (Yield: 0.6 gms)

The PXRD pattern of the above obtained compound was illustrated in FIG. 4.

Example-8: Preparation of amorphous premix of Tafamidis meglumine of formula (Ia) with hydroxypropylmethylcellulose (HPMC).

Tafamidis meglumine of formula (Ia) was dissolved in water (11 ml) at 55-60° C. and added to HPMC solution (0.5 gr of HPMC dissolved in 11 ml of methanol). Distilled off the solvent from the mixture under reduced pressure and then dried to afford the title compound.

Yield: 0.62 gms.

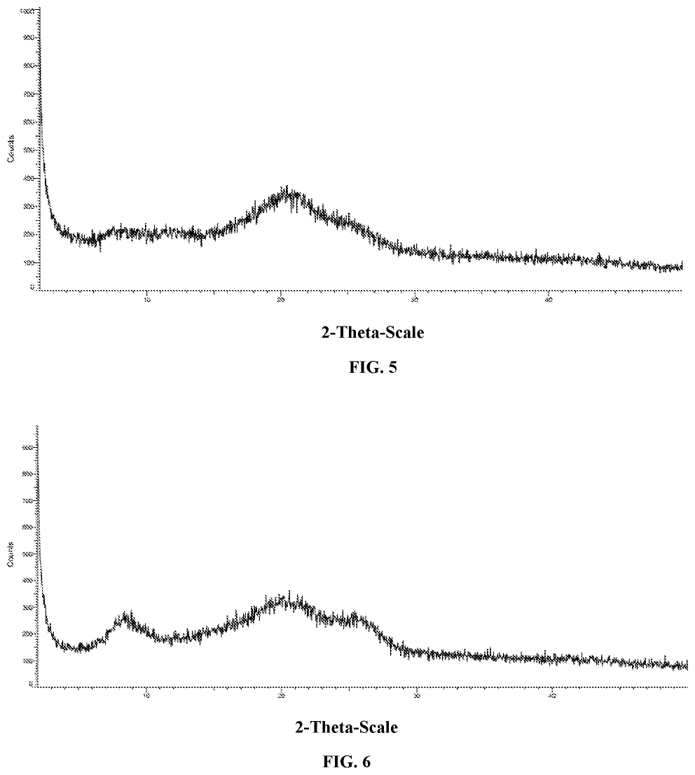

The PXRD pattern of the obtained product was illustrated in FIG. 5.

Example-9: Preparation of amorphous premix of Tafamidis meglumine of formula (Ia) with hydroxy propyl cellulose (HPC).

Tafamidis meglumine of formula (Ia) (0.5 gr) was dissolved in water (10 ml) at 55- 60° C. and stirred for 30 min. Hydroxy propyl cellulose solution (0.5 gr of HPC was dissolved in 10 ml of methanol) was added to the above obtained solution and distilled off the solvent and then dried to get the title compound.

Yield: 0.64 gms

PXRD pattern of the obtained premix of obtained compound was illustrated in FIG. 6.

Example-10: Preparation of amorphous premix of Tafamidis meglumine of formula (Ia) with micro crystalline cellulose (MCC)

Tafamidis meglumine of formula (Ia) (0.5 gr) was dissolved in water (10 ml) at 55- 60° C. and stirred for 30 min. MCC (0.5 gr) was added to the above obtained solution. Distilled off the solvent from the mixture and then dried to get the title compound.

Yield: 0.65 gms

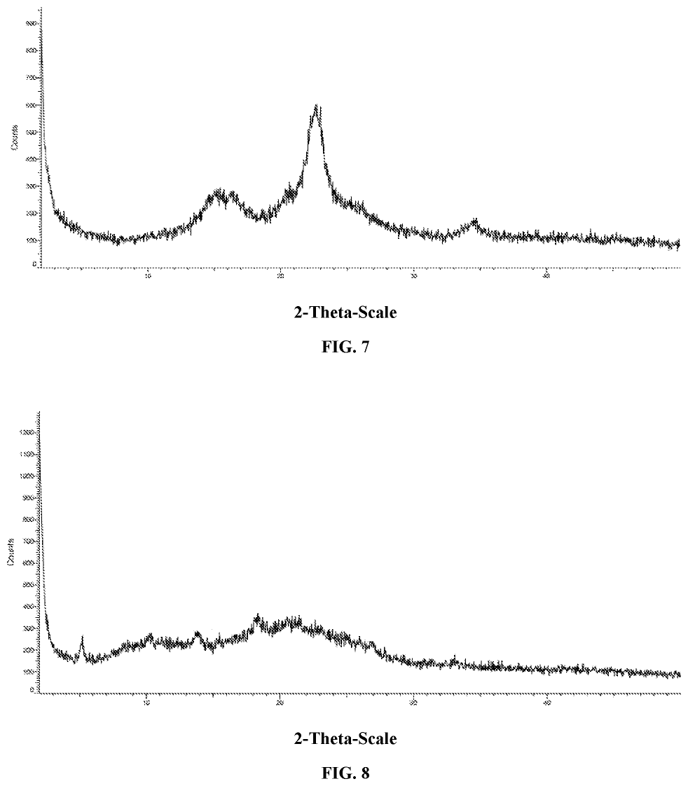

PXRD pattern of the obtained product was illustrated in FIG. 7.

Example-11: Preparation of amorphous premix of Tafamidis meglumine of formula (Ia) with hydroxy propyl methyl cellulose—acetyl succinate (HPMC-AS).

Tafamidis meglumine of formula (Ia) (0.5 gms) was dissolved in water (10 ml) at 55- 60° C. and stirred for 30 min and added to HPMC-AS solution (0.5 gms of HPMC-AS dis- solved in 3 ml methanol). Distilled off the solvent from the mixture and then dried to provide the title compound.

Yield: 0.63 gms

The PXRD pattern of obtained compound was illustrated in FIG. 8.

Example-12: Preparation of amorphous premix of Tafamidis meglumine of formula (Ia) with Syloid.

Tafamidis meglumine of formula (Ia) (0.5 gr) was dissolved in water (10 ml) at 55- 60° C. and stirred for 30 min. Syloid (0.5 gr) was added to the above solution and distilled off the solvent from the mixture and then dried to provide the title compound.

Yield: 0.65 gms

PXRD pattern of the obtained compound was illustrated in FIG. 9.

Example-13: Preparation of amorphous premix of Tafamidis meglumine of formula (Ia) with co-povidone.

Tafamidis meglumine of formula (Ia) (0.5 gms) was dissolved in water (10 ml) at 55- 60° C. and stirred for 30 min and added to aqueous co-povidone solution (0.5 gms of co- povidone dissolved in 3 ml of water). Distilled off solvent from the mixture and dried to get the title compound.

Yield: 0.70 gms

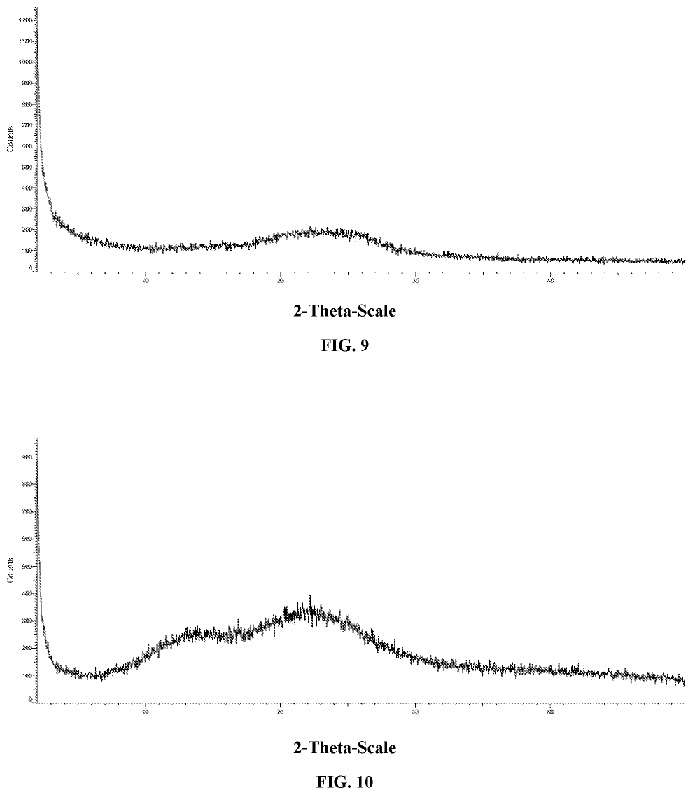

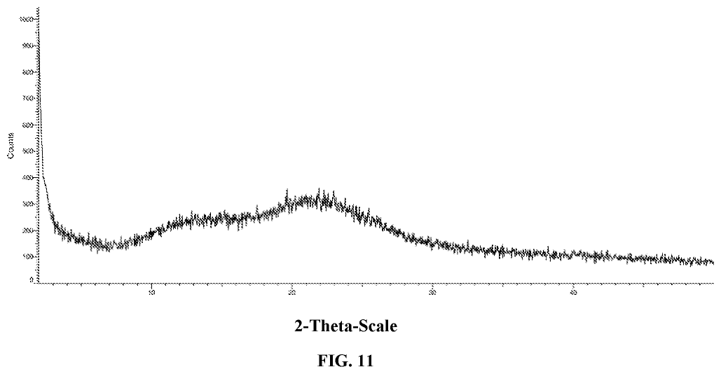

The PXRD pattern of the above obtained compound was illustrated in FIG. 10.

Example-14: Preparation of amorphous form of Tafamidis meglumine of formula (Ia)

Tafamidis meglumine of formula (Ia) (10 gr) was dissolved in water (250 ml) at 70- 75° C. and stirred for 20 min. The solution was filtered and introduced into spray dryer under following conditions:
i) In let temperature: 80° C.
ii) Out let temperature: 100° C.
iii) Nitrogen pressure: 3.0 kg
iv) Aspirator: 70%.

Yield: 6.0 gms

The PXRD pattern of the obtained amorphous form of Tafamidis meglumine was illustrated in FIG. 11.

1. Crystalline Form-R of Tafamidis of formula (I) 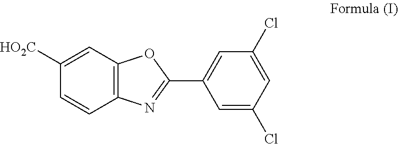 [SMILES: O=C(O)c1ccc2nc(-c3cc(Cl)cc(Cl)c3)oc2c1]
characterized by Powder X-Ray Diffraction (PXRD) pattern having peaks at about 5.2 and 13.7 ±0.2 degrees two-theta.

2. The crystalline Form-R of Tafamidis of formula (I) of claim 1, further characterized by PXRD pattern having additional peaks at about 5.2, 13.7 and 18.0 ±0.2 degrees two-theta.

3. The crystalline Form-R of Tafamidis of formula (I) of claim 1, further characterized by the PXRD pattern as illustrated in FIG. 3.

4. A process for the preparation of crystalline Form-R of Tafamidis of formula (I) of claim 1, comprising drying of crystalline Form-N of Tafamidis of formula (I).

5. The process of claim 4, wherein drying is carried out using vaccum tray dryer, atmospheric tray dryer, rotary cone vacuum dryer, vacuum oven, air oven, fluidized bed dryer, spin flash dryer, flash dryer, and thereof, at atmospheric pressure or under reduced pressure.

6. A process for the preparation of crystalline Form-R of Tafamidis of formula (I), comprising:
a) providing a mixture of Tafamidis of formula (I) in a first solvent.
b) combining the mixture obtained in step-a) with second solvent,
c) isolating crystalline Form-R of Tafamidis of formula (I).

7. The process of claim 6, wherein the first solvent used in step-a) is polar aprotic solvent selected from dimethylsulfoxide, dimethyl formamide, dimethylacetamide and thereof.

8. The process of claim 6, wherein the second solvent used in step-b) is water.

9. The process claim 6, wherein isolating in step-c) carried out by slurrying/stirring the obtained compound with acetic acid and water followed by filtration and drying.

10 - 15. (cancelled).

16. A process for the preparation of Tafamidis of formula (I) or its pharmaceutically acceptable salts, comprising:
a) reacting 3,5-dichlorobenzoyl chloride with 4-amino-3-hydroxybenzoic acid to get 4- (3,5-dichlorobenzamido)-3-hydroxybenzoic acid in the absence of a base;
b) converting 4-(3,5-dichlorobenzamido)-3-hydroxybenzoic acid to Tafamidis formula (I) or its pharmaceutically acceptable salts.

17. (canceled) .

18. Pharmaceutical composition comprising crystalline Form-R of Tafamidis of claim 1 and one or more pharmaceutically acceptable excipients.
